(19) United States
(12) Patent Application Publication
(10) Pub. No.: US 2021/0031047 A1
(43) Pub. Date: Feb. 4, 2021

(54) THERAPY IN THE EVENT OF ELECTRODE FAILURE

(71) Applicant: BIOTRONIK SE & Co. KG, Berlin (DE)

(72) Inventors: THOMAS DOERR, BERLIN (DE); ANDREAS KUCHER, SCHWEDT (DE)

(21) Appl. No.: 16/944,443

(22) Filed: Jul. 31, 2020

(30) Foreign Application Priority Data
Aug. 2, 2019 (EP) 19189736

(51) Int. Cl. A61N 1/39; H02H 3/08; H02H 3/02; A61N 1/05

(52) U.S. Cl. CPC A61N 1/3931; H02H 3/08; A61N 1/3975; A61N 1/0563; A61N 1/3956; H02H 3/021

(57) ABSTRACT

An implantable medical device for the defibrillation of a patient's heart includes an energy storage device for providing a voltage, and at least one electrode for generating an electrical current pulse by way of the voltage. The energy storage device includes at least two capacitors for providing the voltage. The medical device is configured to prompt a further electrical current pulse by way of a reduced voltage from a parallel connection of the at least two capacitors in the event of the short circuit. A method for controlling an implantable medical device is also provided.

### CROSS-REFERENCE TO RELATED APPLICATION

This application claims the priority, under 35 U.S.C. § 119, of European Patent Application EP 19189736, filed Aug. 2, 2019; the prior application is herewith incorporated by reference in its entirety.

### BACKGROUND OF THE INVENTION

### Field of the Invention

The invention relates to an implantable medical device for the defibrillation of a patient's heart and to a method for controlling such a medical device.

Such implantable medical devices are also referred to as implantable cardioverter defibrillators (ICDs for short). Such a medical device is configured to detect ventricular fibrillation of the patient's heart and to treat it by delivering an electric shock or a current pulse.

The problem with an implantable medical device having a defibrillation function is the circumstance that, due to the comparatively high voltage for generating a current pulse, high voltage flashovers may occur in the event of an insulation defect of an electrode, causing a very high fault current. Current ICDs include an electronic short circuit fuse for that failure event, so that the ICD does not experience any component damage in the event of a short circuit, and furthermore is able to carry out the diagnostic and therapeutic functions thereof, however with the limitation that the high voltage therapy remains ineffective.

In that regard, Published U.S. Patent Application 2010228307 describes a reduction of the shocking energy in the ICD when electrode failures are identified so as to prevent a voltage flashover from the electrode failure site to the device housing.

In the event of an identified short circuit in the shock electrode path, it is furthermore known in the prior art to switch the shock electrode configuration or the shock path for subsequent therapy attempts, provided that a so-called dual coil electrode is used. Dual coil electrodes include a right ventricular shock coil (RV coil) and a second shock coil in the superior vena cava (superior vena cava coil, SVC coil). Different shock paths between the RV coil, the SVC coil and the implant housing can be selected. In that regard, refer to Swerdlow, et al, “Implantable Cardiac Defibrillator Lead Failure and Management,” in the JOURNAL OF THE AMERICAN COLLEGE OF CARDIOLOGY, VOL. 67, NO. 11, 2016.

However, the absence of a high voltage therapy due to short circuit protection is not desirable since the patient may thus possibly not be provided with a necessary therapy. In principle, it is desirable to be able to provide the patient with a high voltage therapy/defibrillation even in the above-described failure case, which in particular is not limited by the use of a specific shock electrode.

### BRIEF SUMMARY OF THE INVENTION

It is accordingly an object of the invention to provide an implantable medical device and a method for controlling an implantable medical device for therapy in the event of an electrode failure, which overcome the hereinafore-mentioned disadvantages of the heretofore-known devices and methods of this general type and which provide an implantable medical device for the defibrillation of a patient's heart which, in the event of an insulation defect of a defibrillation electrode with short circuit identification and triggering of the short circuit fuse in the device, is nonetheless able to carry out further therapy attempts, which have an increased likelihood of being successful in this situation for terminating life-threating ventricular tachyarrhythmia.

With the foregoing and other objects in view there is provided, in accordance with the invention, an implantable medical device for the defibrillation of a patient's heart, comprising:

an energy storage device, which includes at least two capacitors for providing a voltage;

at least one electrode for delivering an electrical current pulse by way of the voltage; and

a short circuit fuse, which is configured to be triggered in the event of a short circuit of the at least one electrode and to terminate or to prevent a delivery of an electrical current pulse, wherein the implantable medical device is configured, in the event of the short circuit, to prompt the delivery of a further current pulse, in particular by way of a reduced voltage, from a parallel connection of the at least two capacitors.

A reduced voltage shall be understood to mean here that the further current pulse is delivered with a voltage that is reduced compared to the voltage of the terminated or prevented current pulse.

The invention thus allows an adapted high voltage therapy attempt (defibrillation) to be delivered in the event that an electrode defect is present in the high voltage therapy path, by preferably reducing the therapy voltage, in the event that an electrode failure is identified, in such a way that a higher likelihood of the therapy being successful is achieved in this situation.

The approach according to the invention has the advantage that the therapy or shock voltage can be reduced compared to that of a normal defibrillation shock, while the delivered shocking energy substantially remains the same.

The invention can, in particular, utilize the mechanism of a typical short circuit in the defibrillation path of the device. During an insulation defect of the electrode used for shocking, in general no purely metallic contact exists between the high voltage-conducting conductors or components, but a voltage flashover or arc occurs at the moment the high voltage is delivered. The likelihood of such a flashover increases with the applied maximum voltage, and can therefore be reduced if the maximum voltage is reduced in subsequent therapy attempts, however only to the extent that sufficient therapy success continues to be possible.

According to a preferred embodiment of the implantable medical device according to the invention, it is further provided that the implantable medical device includes an identification device, which is configured to identify the triggering of the short circuit fuse, and to signal this to a control unit of the implantable medical device, wherein the control unit is configured to deliver the further current pulse having the preferably reduced voltage if it has been identified that the short circuit fuse was triggered.

According to one embodiment of the invention, it is further provided that the control unit is configured to prompt a switch of a series connection of capacitors to the parallel connection of the at least two capacitors if it has been identified that the short circuit fuse was triggered. For example, an overall voltage present at the two capacitors connected in series can be reduced in the process to a lower voltage that is present at the capacitors now connected in parallel.

According to a preferred embodiment of the implantable medical device according to the invention, it is further provided that the at least one electrode of the implantable medical device is configured to be inserted into the ventricle of the patient via a vein (that is, the medical device is a so-called transvenous cardioverter defibrillator).

According to a preferred embodiment of the implantable medical device according to the invention, it is further provided that the at least one electrode of the implantable medical device is configured to be subcutaneously implanted, and more particularly above the patient's sternum.

According to a preferred embodiment of the implantable medical device according to the invention, it is further provided that the at least one electrode of the implantable medical device is configured to be substernally implanted (that is, under the patient's sternum).

The implantable medical device preferably includes a housing for accommodating the at least two capacitors or the energy storage device, as well as in particular the further components (such as the short circuit fuse and/or the control unit and/or identification device). The at least one electrode is connected to the housing.

In the case of a subcutaneous or transvenous electrode, the housing can be configured, for example, to be implanted into the patient's chest (for example, on the left next to the thorax, below the axilla). The housing can also be implanted in the patient's chest in the case of a substernal electrode.

With the objects of the invention in view, there is concomitantly provided a method for controlling an implantable medical device (in particular an implantable medical device according to the invention), comprising the following steps:

providing a voltage by way of at least two capacitors of the implantable medical device for delivering a current pulse; and

terminating or preventing a delivery of the electrical current pulse in the event a short circuit of an electrode of the implantable medical device is identified; and

delivering a further current pulse from a parallel connection of the at least two capacitors.

According to a preferred embodiment of the method according to the invention, it is provided that a short circuit fuse of the implantable medical device is triggered in the event of a short circuit of an electrode, and a delivery of an electrical current pulse by way of the implantable medical device is thereby terminated or prevented, wherein, in particular in the case of a short circuit, the further current pulse is delivered from the parallel connection of the at least two capacitors.

According to a preferred embodiment of the method according to the invention, it is further provided that the triggering of the short circuit fuse is automatically identified by way of an identification device, wherein the identification device signals the triggering of the short circuit fuse to a control unit of the implantable medical device, and wherein the control unit prompts the delivery of the further current pulse if it has been identified that the short circuit fuse was triggered.

According to a preferred embodiment of the method according to the invention, it is provided that a switch of a series connection of the capacitors to the parallel connection of the at least two capacitors is automatically prompted by the implantable medical device in the event of a short circuit (for example, by the control unit), and thereafter the delivery of the further current pulse is prompted.

Other features which are considered as characteristic for the invention are set forth in the appended claims.

Although the invention is illustrated and described herein as embodied in an implantable medical device and a method for controlling an implantable medical device for therapy in the event of an electrode failure, it is nevertheless not intended to be limited to the details shown, since various modifications and structural changes may be made therein without departing from the spirit of the invention and within the scope and range of equivalents of the claims.

The construction and method of operation of the invention, however, together with additional objects and advantages thereof will be best understood from the following description of specific embodiments when read in connection with the accompanying drawings.

### BRIEF DESCRIPTION OF THE SEVERAL VIEWS OF THE DRAWING

FIG. 1 is a schematic and block diagram of a typical failure mechanism of an implantable medical device, which can result in a short circuit of an electrode of the device;

FIG. 2 is a diagram showing a reduction of the voltage of the medical device for generating an electrical current pulse for a defibrillation;

FIG. 3 is a diagram showing a switch between a series connection of capacitors of the implantable medical device and a parallel connection of the capacitors for reducing the voltage; and

FIG. 4 is a diagram showing a further option for reducing the voltage for the defibrillation.

### DETAILED DESCRIPTION OF THE INVENTION

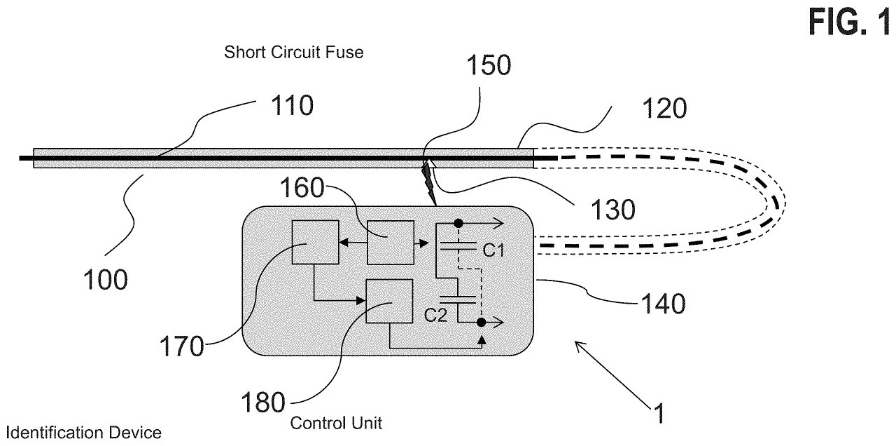

Referring now to the figures of the drawings in detail and first, particularly, to FIG. 1 thereof, there is seen a typical failure mechanism of an insulation defect during a defibrillation shock delivery (that is, delivery of an electrical current pulse) by an implantable medical device 1 according to the invention. At least one electrode 100 is implemented as an electrode lead 100 and includes, in particular, an electrical conductor 110 and an insulator 120 surrounding the same, wherein this insulator 120 has an insulation defect 130 in the event of a failure. The counter electrode for the defibrillation is a housing 140 of the implantable medical device 1 or defibrillator 1 in this case.

Since, in this system, the electrical conductor 110 and the housing 140 typically cannot touch one another even in the area of the insulation defect 130, an identification of this insulation defect is not possible by way of a low voltage measurement, that is, the so-called painless shock impedance measurement does not show any abnormality here.

If, however, a high voltage therapy is now delivered by way of the electrode 100 and the counter electrode 140, a high voltage flashover 150 may occur in the area of the insulation defect, resulting in a very high fault current. The implantable electronic device 1 therefore includes an electronic short circuit fuse 160 for this failure event, so that the device 1 does not experience any component damage in the event of a short circuit, and continues to be able to able to carry out the diagnostic and therapeutic functions thereof, however with the limitation that the high voltage therapy remains ineffective.

The short circuit fuse 160 is configured to be triggered in the event of a short circuit of the at least one electrode 100 and to terminate or to prevent a delivery of an electrical current pulse by the implantable medical device 1. The implantable medical device 1 further preferably includes an identification device 170 (refer to FIG. 1), which is configured to identify the triggering of the short circuit fuse 160 and to signal this to a control unit 180 of the implantable medical device 1, wherein the control unit 180 is configured to prompt a further electrical current pulse from a parallel connection of at least two capacitors C1, C2 if it has been identified that the short circuit fuse 160 was triggered. According to one embodiment of the invention, the control unit 180 or the medical device 1 is configured to carry out a switch from a previous series connection of the capacitors C1, C2 to a parallel connection (dotted line between C1 and C2 in FIG. 1) in such a way that the delivery of the further current pulse from the parallel connection takes place with a voltage that is reduced compared to the voltage of the series connection of the capacitors intended with the terminated or with the prevented current pulse.

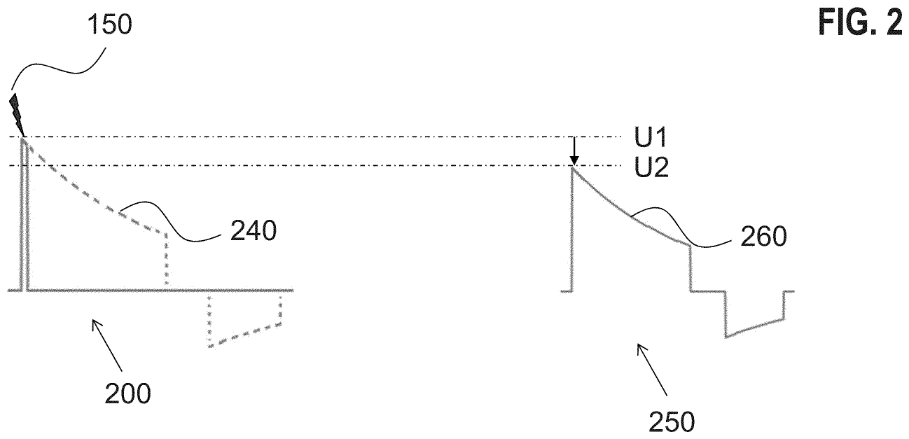

FIG. 2 shows a schematic representation of the reduction of the voltage according to the invention during the delivery of the shock therapy in the event of an insulation defect. A first delivered defibrillation shock or electrical current pulse 200 is delivered with a programmed or maximum voltage U1, which causes a high voltage flashover 150 in the area of the insulation defect 130, and the shock delivery is thereupon terminated by the electronic short circuit fuse 160. A shocking energy 240 to be delivered thus does not become effective for the defibrillation.

According to the invention, in this implementation example, the maximum voltage is set to a reduced value U2 (in particular by the above-described switch of the capacitors C1 and C2) during the next defibrillation attempt 250, after it has been identified that the short circuit fuse 160 was triggered, so that the likelihood of a renewed high voltage flashover is reduced, while it is still possible to deliver sufficient shocking energy 260 for likely success of the therapy. If, in this case, no renewed high voltage flashover occurs, the intended defibrillation energy is delivered, and the tachyarrhythmia can thus be terminated.

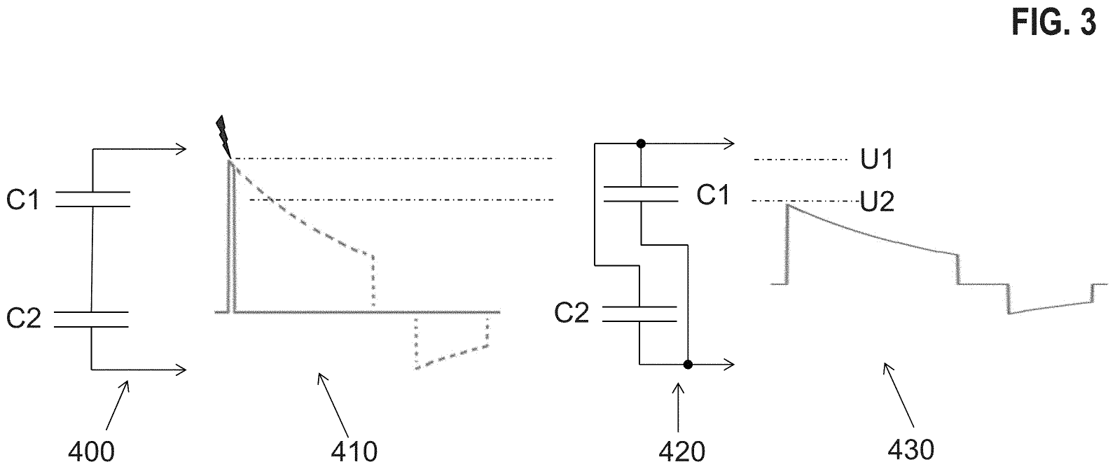

FIG. 3 schematically illustrates the above-described switch of the capacitors C1, C2. Initially, a defibrillation shock 410 or electrical current pulse is delivered from a series connection 400 of the two capacitors C1, C2, which in the event of a failure causes the short circuit fuse 160 to be triggered (see above). A subsequent shock is now delivered from a parallel connection 420 of the two capacitors C1, C2, which offers the advantage that the shock peak voltage is cut in half, for example, and at the same time substantially the entire maximum shocking energy 430 can be delivered.

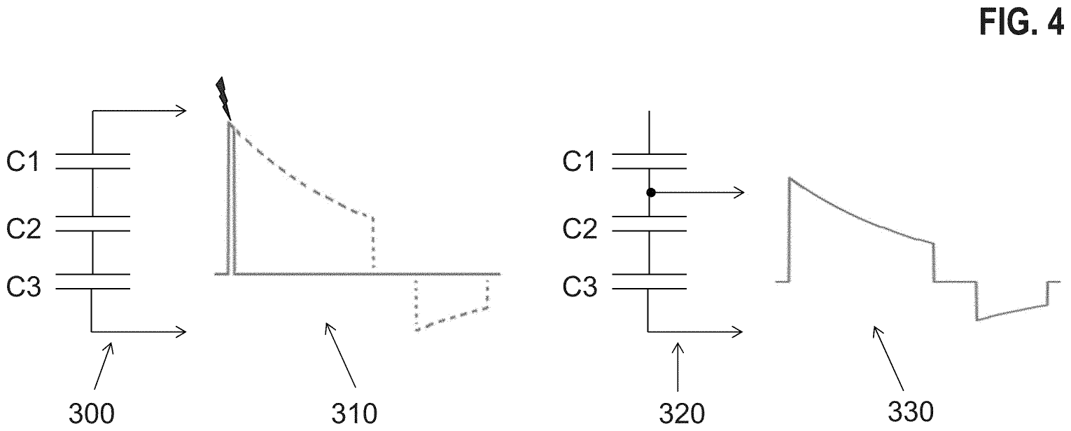

FIG. 4 illustrates another option of reducing the voltage so as to implement a defibrillation shock delivery with a considerably reduced peak voltage, and thereby reduce the likelihood of a high voltage flashover.

A first shock 310 or electrical current pulse for defibrillation is delivered from a series connection 300 of three capacitors C1, C2, C3 here, wherein this results in the short circuit fuse 160 being triggered as a result of an insulation defect.

In the subsequent shock or current pulse 330, the tap for the shock delivery is only set in a series connection 320 of the capacitors C2, C3 by way of electronic switch elements (not shown here), and the peak voltage of the shock 330 is thereby reduced by ⅓. In this system, the capacitance resulting during the shock delivery increases, and the duration of the shock phases is thus extended, which can result in a higher efficiency of the therapy, yet also a lower shock peak voltage, if the capacitances are suitable configured.

The invention advantageously increases the likelihood that a life-saving therapy can still be delivered in the event of an electrode defect of an implantable medical device.

It will be apparent to those skilled in the art that numerous modifications and variations of the described examples and embodiments are possible in light of the above teaching. The disclosed examples and embodiments are presented for purposes of illustration only. Other alternate embodiments may include some or all of the features disclosed herein. Therefore, it is the intent to cover all such modifications and alternate embodiments as may come within the true scope of this invention.

1. An implantable medical device for the defibrillation of a patient's heart, the implantable medical device comprising:
an energy storage device including at least two capacitors for providing a voltage;
at least one electrode for delivering an electrical current pulse by way of the voltage; and
a short circuit fuse configured to be triggered upon an occurrence of a short circuit of said at least one electrode and to terminate or to prevent a delivery of an electrical current pulse;
said at least two capacitors being connected in parallel for delivering a further current pulse upon the occurrence of the short circuit.

2. The implantable medical device according to claim 1, which further comprises a control unit, and an identification device configured to identify the triggering of said short circuit fuse and to signal the triggering of said short circuit fuse to said control unit, said control unit being configured to prompt the delivery of the further current pulse upon the identification that said short circuit fuse was triggered.

3. The implantable medical device according to claim 2, wherein said control unit is configured to prompt a switch of a series connection of said capacitors to said parallel connection of said at least two capacitors upon the identification that said short circuit fuse was triggered.

4. The implantable medical device according to claim 1, wherein said at least one electrode is configured to be inserted into a ventricle of the patient via a vein.

5. The implantable medical device according to claim 1, wherein said at least one electrode is configured to be subcutaneously implanted.

6. The implantable medical device according to claim 1, wherein said at least one electrode is configured to be substernally implanted.

7. A method for controlling an implantable medical device, the method comprising the following steps:
providing a voltage from at least two capacitors of the implantable medical device for delivering a current pulse; and
terminating or preventing a delivery of the electrical current pulse upon an identification of a short circuit of an electrode of the implantable medical device; and
delivering a further current pulse from a parallel connection of the at least two capacitors.

8. The method according to claim 7, which further comprises triggering a short circuit fuse of the implantable medical device upon an occurrence of the short circuit of the electrode, and thereby terminating or preventing a delivery of the electrical current pulse.

9. The method according to claim 8, which further comprises:
using an identification device to identify the triggering of the short circuit fuse;
using the identification device to signal the triggering of the short circuit fuse to a control unit of the implantable medical device; and
using the control unit to cause the delivery of the further current pulse upon the identification that the short circuit fuse was triggered.

10. The method according to claim 9, which further comprises using the control unit to cause a switch of a series connection of capacitors to the parallel connection of the at least two capacitors upon the identification that the short circuit fuse was triggered.
